(19) United States
(12) Patent Application Publication
(10) Pub. No.: US 2018/0353278 A1
(43) Pub. Date: Dec. 13, 2018

(54) PROSTHETIC PHRENOESOPHAGEAL MEMBRANE

(71) Applicant: Swedish Health Services, Seattle, WA (US)

(72) Inventor: Ralph William Aye, Seattle, WA (US)

(73) Assignee: Swedish Health Services, Seattle, WA (US)

(21) Appl. No.: 15/771,795

(22) PCT Filed: Nov. 4, 2016
(86) PCT No.: PCT/US2016/060721

§ 371 Date: Apr. 27, 2018

Related U.S. Application Data
(60) Provisional application No. 62/251,271, filed on Nov. 5, 2015.

(51) Int. Cl. A61F 2/00; A61F 2/04; A61L 31/10; A61L 31/04

(52) U.S. Cl. CPC A61F 2/0063; A61F 2/04; A61L 31/10; A61F 2002/044; A61F 2002/0068; A61F 2230/0067; A61F 2250/0039; A61L 31/048

(57) ABSTRACT

Current surgical techniques to repair hiatal hernias including pulling the stomach down and out of the hiatus to reposition it properly on the posterior side of the diaphragm, and patching the hiatus and or applying basic mesh to repair the hiatus and esophageal membrane. However, despite these techniques, the anatomic hernia recurrence rate for hiatal hernia repair is over 50% at 5 years. Accordingly, the present disclosure discloses a new funnel shaped mesh that can be implanted during hiatal hernia repair surgery that would function effectively as a phrenoesophageal membrane and reduce recurrence rates dramatically. The mesh could have the narrower portion attach to the esophagus, while the larger diameter funnel top could adhere to the diaphragm.

### FIELD OF THE DISCLOSURE

The present disclosure is directed to prosthetic reinforcement of the esophageal hiatus. More particularly, the present disclosure relates to a device, a method, and a system for repair of a paraesophagel hernia utilizing a funnel shaped mesh that effectively functions as a phrenoesophageal membrane while reducing recurrence rates of paraesophagel hernia.

### BACKGROUND OF THE DISCLOSURE

The following description includes information that may be useful in understanding the present disclosure. It is not an admission that any of the information provided herein is prior art or relevant to the presently claimed disclosure, or that any publication specifically or implicitly referenced is prior art.

Hiatal hernias essentially are the result of the protrusion of the stomach through the hiatus of the diaphragm. The hiatus is an opening in the diaphragm—the muscular wall separating the chest cavity from the abdomen. Normally, the esophagus passes through the hiatus and connects to the stomach, with the stomach situated entirely on the posterior or underside of the diaphragm. However, in a paraesophageal hernia which is a type of hiatal hernia, the stomach bulges up through the hiatal opening into the chest cavity on the anterior side of the diaphragm. Paraesophageal hernia can result in the obstruction of the stomach or ischemia of the blood supply to the stomach and can thus have serious consequences.

Hiatal hernias are currently repaired using laparoscopic surgery. In this procedure, the stomach is pulled back down through the hiatus and below the diaphragm. After resituating the stomach, the surgeon usually attempts to close the diaphragm at the hiatus to prevent re-herniation. After closure, a biological or permanent mesh patch may be added posteriorly over the hiatal closure. Other less effective techniques that may be used are relaxing incision in the diaphragm.

Despite these techniques, the anatomic hernia recurrence rate for hiatal hernia repair is over 50% at five years and beyond, using current surgical techniques. For instance, even though biologic mesh has been used, currently it has not been shown to reduce long term recurrences.

### SUMMARY OF THE DISCLOSURE

Disclosed herein are device and methods for repair of a paraesophagel hernia utilizing a funnel shaped mesh. This prosthetic reinforcement of the esophageal hiatus is intended to be implanted at the time of hiatal hernia repair and/or anti-reflux surgery. It consists of a mesh placed as a collar around the esophagus and extending onto the diaphragm.

This prosthetic may include a thin delicate permanent mesh which may conform to the esophagus with minimal risk of erosion, and replace or augment the natural phrenoesophageal membrane bridging the space between the esophagus and the diaphragm which is lost with hiatal hernias.

The device may include an open funnel shape, with the lower three quarters or other proportion encircling the esophagus except along the lesser curvature of the stomach. This lower three quarters may be include a lightweight mesh with a roughened undersurface to adhere to the esophagus. The upper quarter or other proportion of the funnel may include a stiffer mesh to give it a circular shape and allow it to be anchored to the diaphragm. The open part or break in the funnel allows a surgeon to implant the funnel around the esophagus by wrapping it around before attachment. In other embodiments, the mesh may only be a partial funnel and only encircle, half, three quarters, or one quarter or other suitable percentage of the esophagus.

The outer surface of the disclosed device may be covered by biologic material to reduce adherence of the stomach and surrounding tissue, and to reduce the risk of erosion. The mesh may include polypropylene on one side and PTFE on the other side or another biologic to prevent the stomach from adhering to it.

### BRIEF DESCRIPTION OF THE DRAWINGS

The accompanying drawings, which are incorporated in and constitute a part of this specification, exemplify the embodiments of the present disclosure and, together with the description, serve to explain and illustrate principles of the disclosure. The drawings are intended to illustrate major features of the exemplary embodiments in a diagrammatic manner. The drawings are not intended to depict every feature of actual embodiments nor relative dimensions of the depicted elements, and are not drawn to scale.

FIG. 1 illustrates an example of a perspective view of a funnel mesh for paraesophageal hernia repair that is constructed in accordance with the principles of the present disclosure.

FIG. 2 illustrates an example of a perspective view of a funnel mesh implanted in a patient to repair a paraesophageal hernia that is constructed in accordance with the principles of the present disclosure.

FIG. 3 illustrates an example of a method for implanting a funnel mesh in accordance with the principles of the present disclosure.

In the drawings, the same reference numbers and any acronyms identify elements or acts with the same or similar structure or functionality for ease of understanding and convenience. To easily identify the discussion of any particular element or act, the most significant digit or digits in a reference number refer to the Figure number in which that element is first introduced.

### DETAILED DESCRIPTION

Unless defined otherwise, technical and scientific terms used herein have the same meaning as commonly understood by one of ordinary skill in the art to which this disclosure belongs. Szycher's Dictionary of Medical Devices CRC Press, 1995, may provide useful guidance to many of the terms and phrases used herein. One skilled in the art will recognize many methods and materials similar or equivalent to those described herein, which could be used in the practice of the present disclosure. Indeed, the present disclosure is in no way limited to the methods and materials specifically described.

In some embodiments, properties such as dimensions, shapes, relative positions, and so forth, used to describe and claim certain embodiments of the disclosure are to be understood as being modified by the term “about.”

Various examples of the disclosure will now be described. The following description provides specific details for a thorough understanding and enabling description of these examples. One skilled in the relevant art will understand, however, that the disclosure may be practiced without many of these details. Likewise, one skilled in the relevant art will also understand that the disclosure can include many other obvious features not described in detail herein. Additionally, some well-known structures or functions may not be shown or described in detail below, so as to avoid unnecessarily obscuring the relevant description.

The terminology used below is to be interpreted in its broadest reasonable manner, even though it is being used in conjunction with a detailed description of certain specific examples of the disclosure. Indeed, certain terms may even be emphasized below; however, any terminology intended to be interpreted in any restricted manner will be overtly and specifically defined as such in this Detailed Description section.

While this specification contains many specific implementation details, these should not be construed as limitations on the scope of any disclosures or of what may be claimed, but rather as descriptions of features specific to particular implementations of particular disclosures. Certain features that are described in this specification in the context of separate implementations can also be implemented in combination in a single implementation. Conversely, various features that are described in the context of a single implementation can also be implemented in multiple implementations separately or in any suitable subcombination. Moreover, although features may be described above as acting in certain combinations and even initially claimed as such, one or more features from a claimed combination can in some cases be excised from the combination, and the claimed combination may be directed to a subcombination or variation of a subcombination.

Similarly while operations may be depicted in the drawings in a particular order, this should not be understood as requiring that such operations be performed in the particular order shown or in sequential order, or that all illustrated operations be performed, to achieve desirable results. In certain circumstances, multitasking and parallel processing may be advantageous. Moreover, the separation of various system components in the implementations described above should not be understood as requiring such separation in all implementations, and it should be understood that the described program components and systems can generally be integrated together in a single software product or packaged into multiple software products.

### Overview

Disclosed herein are devices and methods for repairing hiatal hernias including a funnel shaped mesh. The hiatus, the orifice in the diaphragm through which the esophagus passes to connect to the stomach, is surrounded by a phrenoesophageal membrane that prevents the esophagus from sliding upwards through the hiatus, and prevents the esophagus from dragging the stomach through the hiatus. This prevents the stomach from protruding through the hiatus and entering the anterior side of the diaphragm. However, sometimes the stomach slides through the membrane, bulges through the hiatus and does enter the anterior side of the stomach, resulting in a hiatal hernia.

Current surgical techniques to repair hiatal hernias includes pulling the stomach down and out of the hiatus to reposition it properly on the posterior side of the diaphragm, and patching the hiatus and or applying basic mesh to repair the hiatus and esophageal membrane. However, despite these techniques, the anatomic hernia recurrence rate for hiatal hernia repair is over 50% at 5 years. Accordingly, the inventors have developed a new funnel shaped mesh that can be implanted during hiatal hernia repair surgery that would function effectively as a phrenoesophageal membrane and reduce recurrence rates dramatically. The mesh could have the narrower portion attach to the esophagus, while the larger diameter funnel top could adhere to the diaphragm.

### Funnel

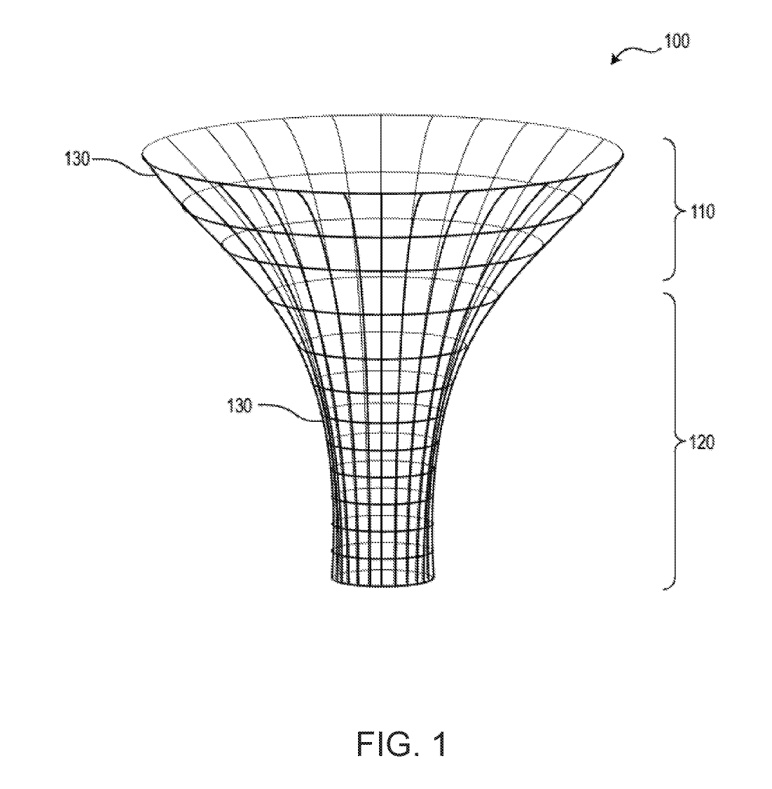

FIG. 1 illustrates an example of a funnel shaped mesh 100 for repair of hiatal hernias that is constructed in accordance with the principles of the present disclosure. The mesh 100 may include two parts: a diaphragm portion 110 and an esophageal portion 120. The diaphragm portion 110 may include a large diameter portion or mouth of the funnel 100. The diaphragm portion 110 may be configured and sized to attach to a diaphragm wall of a patient. For instance, an end of the diaphragm portion 110 may fan out so that it can be attached to the diaphragm wall which is perpendicular to the esophagus.

The esophageal portion 120 may be configured to connect to the diaphragm portion 110 and be sized and configured to attach to the esophagus without restricting bolus movement through the esophagus. Accordingly, the esophageal portion 120 may be sized to fit entirely or partially wrapped around a patient's esophagus. Additionally, the esophageal portion 130 will begin to fan out into the diaphragm portion 110 at an angle that is appropriate for maintaining maximum resistance to migration of the esophagus/stomach back up through the hiatus.

The esophageal portion 120 and diaphragm portion 110 will be connected to form a relatively continuous mesh funnel 100. However, both the esophageal portion 120 and diaphragm portion 110 may include an opening 130 so that the funnel 100 can be wrapped around the esophagus for implantation and attachment to the diaphragm.

### Materials

The esophageal portion 120 may include a material that is relatively more delicate and conforming than the diaphragm portion 110 to allow for the esophageal portion 120 to conform to the esophagus and avoid restricting of peristalsis or bolus flow. Accordingly, in some embodiments, the esophageal portion 120 may include a thin delicate permanent mesh. In some embodiments, the esophageal portion 120 may have elastic properties to allow for peristalsis. For instance, the esophageal portion 120 may include a material, such as, for example, polypropylene, and the like. In some embodiments, the inside or esophagus containing part of the esophageal portion may include a roughened surface to promote adhesion to the esophagus. The outside or outer surface of the esophageal portion 120 may be coated with or include polytetrafluoroethylene (ePTFE) or another biologic material to prevent adhesion to the stomach. For instance, ePTFE microporous expanded film or porous low density polyethylene (LDPE) film may be laminated to the outside of the polypropylene mesh.

The diaphragm portion 110 may include a stiffer mesh (for example, a thicker gage mesh) to facilitate holding a circular/funnel shape, retain resistance to migration of the stomach and esophagus, and allow it to be anchored to the diaphragm. For instance, the esophageal portion 120 may include polypropylene. The stiffer material is permissible because peristalsis restriction is not an issue when attaching to the diaphragm. The outside or outer surface of the diaphragm portion 120 may be coated with or include polytetrafluoroethylene (ePTFE) or another biologic material to prevent adhesion to the stomach.

### Methods

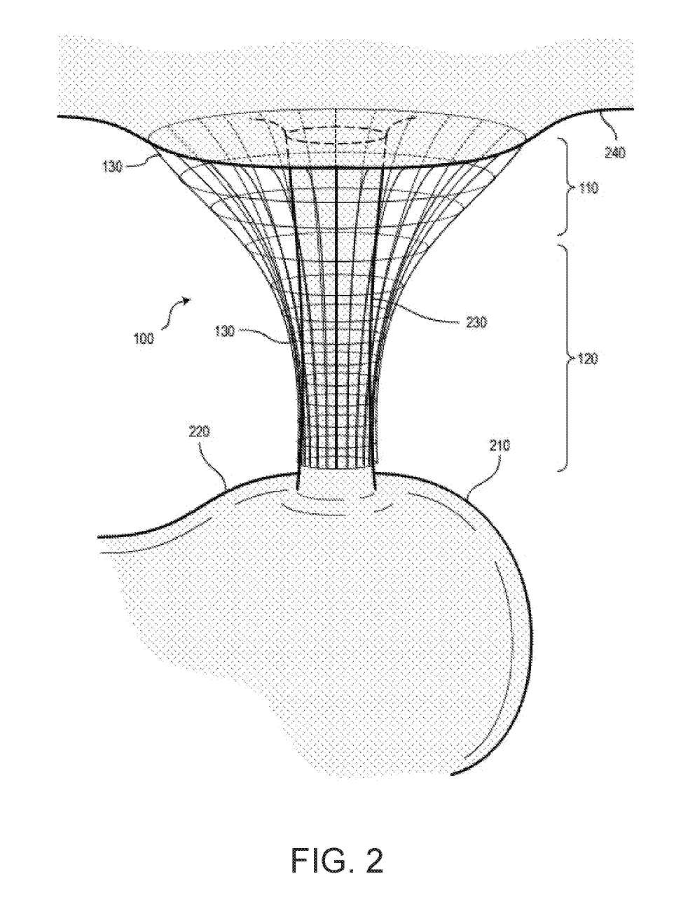

FIG. 2 illustrates an example of an implanted funnel 100, that a surgeon attached after pulling the stomach and esophagus down and out of the hiatus and back on the posterior side of the diaphragm in accordance with the principles of the present disclosure. As shown, a surgeon has wrapped the esophageal portion 120 around a patient's esophagus 230 by wrapping the mesh so that the opening or seam 130 closes together. Then, a surgeon may either affix the esophageal portion 120 directly to the esophagus 230 or the two sides that form the opening 130 may be wrapped to overlap, and then fixed to each other. In some embodiments, a fastening mechanism may be included for quickly affixing and/or adjusting the circumference of funnel 100. For instance, funnel sides that form the opening 130 may include hooks, clips, or other fasteners, or may be fastened with suturing.

A surgeon may also affix the diaphragm portion 110 to a patient's diaphragm 240 either at the same time, prior to, or after fastening the esophageal portion 120. In some embodiments, the diaphragm portion 110 may be sutured or fixed using other methods to a patient's diaphragm 240. To fix the diaphragm portion 110, the surgeon may wrap the diaphragm portion 110 around the esophagus 230 and position it so a portion of the diaphragm portion 110 is flat against the diaphragm 240 for suturing and affixing. The positioning of the diaphragm portion 110 will help for a funnel shaped funnel 100 and may be optimized for maximum retention.

For example, minimizing the arc radius of curvature may prevent the structure from buckling under pressure of the esophagus and stomach migrating upwards towards the hiatus. Accordingly, the surgeon may decide to affix the diaphragm portion using the smallest possible radius.

In some embodiments, the esophageal portion 120 and the diaphragm portion 110 may be separate pieces of different sizes that may be attached and mixed and matched during surgery to select the optimal combination. In these embodiments, the connection between the diaphragm portion 110 and the esophageal portion 120 may include attachment means, such as, for example, hooks, suture, or other attachment methods. In these embodiments, the esophageal portion 120 and the diaphragm portion 110 may be implanted separately and then joined, or may first be joined prior to implantation.

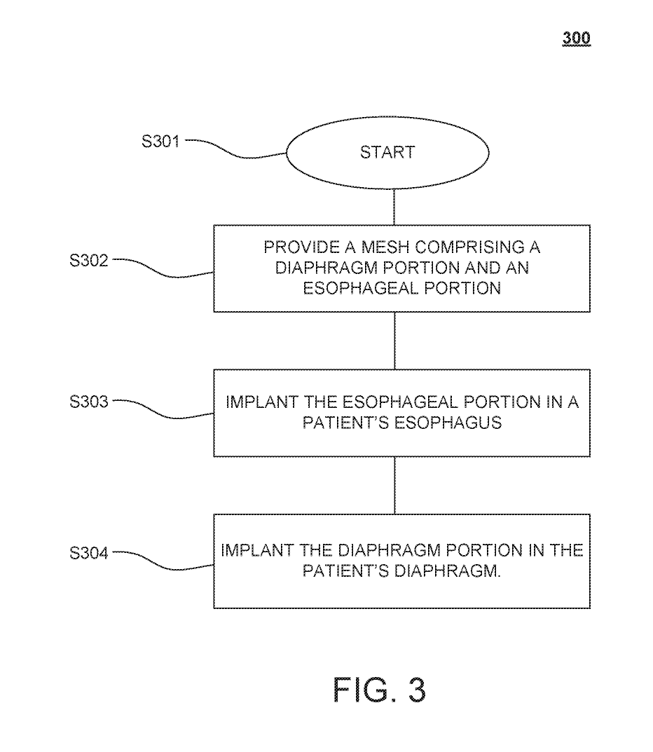

FIG. 3 illustrates an example of a funnel mesh implanted in a patient to repair a paraesophageal hernia that is in accordance with the principles of the present disclosure. The method includes providing a mesh wherein the mesh includes a diaphragm portion and an esophageal portion (S302); implanting the esophageal portion in the patient's esophagus (S303); and implanting the diaphragm portion in the patient's diaphragm (S304).

### SELECTED EMBODIMENTS

Embodiment 1. A surgical mesh for the repair of hiatal hernias comprising: a diaphragm portion sized for attachment to a diaphragm and comprises a large diameter component of a funnel; an esophageal portion connected to the diaphragm portion, wherein the esophageal portion is sized for attachment to an esophagus and sized to form a narrow diameter component of a funnel; and an opening that extends along the entire length of the diaphragm portion and the esophageal portion.

Embodiment 2. The surgical mesh of embodiment 1, wherein the diaphragm portion comprises a heavier gage material than the esophageal portion.

Embodiment 3. The surgical mesh of embodiment 1, wherein the diaphragm portion comprises a stiffer material than the esophageal portion.

Embodiment 4. The surgical mesh of embodiment 1, wherein the diaphragm portion and the esophageal portion are coated on one side with ePTFE.

Embodiment 5. The surgical mesh of embodiment 4, wherein the diaphragm portion and the esophageal portion are coated on other side with ePTFE.

Embodiment 6. The surgical mesh of embodiment 1, wherein the esophageal portion is configured to conform to the esophagus and be flexible enough to allow for peristalsis.

Embodiment 7. The surgical mesh of embodiment 1, wherein the esophageal portion and the diaphragm portion comprises polypropylene.

Embodiment 8. The surgical mesh of embodiment 1, wherein the esophageal portion comprises a rough texture on the side of the mesh that contacts the esophagus.

Embodiment 9. A method for implanting a mesh in a patient to address a hiatal hernia comprising: providing a mesh comprising: a diaphragm portion sized for attachment to a diaphragm and comprises a large diameter component of a funnel; an esophageal portion connected to the diaphragm portion, wherein the esophageal portion is sized for attachment to the patient's esophagus and sized to form a narrow diameter component of a funnel; and an opening that extends along the entire length of the diaphragm portion and the esophageal portion, implanting the esophageal portion by wrapping the esophageal portion around the esophagus and thereby fixing the esophageal portion in place; and implanting the diaphragm portion by positioning the diaphragm portion adjacent to the patient's diaphragm in an orientation that forms a funnel and fixing the esophageal portion in place.

Embodiment 10. The method of embodiment 9, wherein the implanting of the esophageal portion is performed separately from the implanting of the diaphragm portion.

Embodiment 11. The method of embodiment 10, wherein the esophageal portion and diaphragm portions are connected to each other after implantation.

Embodiment 12. The method of embodiment 9, wherein the esophageal portions and diaphragm portions are connected prior to implantation.

Embodiment 13. The method of embodiment 9, wherein the esophageal portion and the diaphragm portion comprises polypropylene.

Embodiment 14. A funnel shaped mesh for repairing hiatal hernias comprising: a diaphragm portion configured for attachment to a diaphragm and sized to form a funnel mouth; an esophageal portion connectable to the diaphragm portion, wherein the esophageal portion is sized for attachment to an esophagus and sized to form a tube part of the funnel; and a seam that extends along the entire length of the diaphragm portion and the esophageal portion that is configured to allow the esophageal portion and the diaphragm portion to be wrapped around an esophagus, wherein the diaphragm portion and the esophageal portion comprise a mesh material.

Embodiment 15. The surgical mesh of embodiment 14, wherein the diaphragm portion comprises a heavier gage material than the esophageal portion.

Embodiment 16. The surgical mesh of embodiment 14, wherein the diaphragm portion comprises a stiffer material than the esophageal portion.

Embodiment 17. The surgical mesh of embodiment 14, wherein the mesh material is coated on one side with ePTFE.

Embodiment 18. The surgical mesh of embodiment 14, wherein the esophageal portion is constructed to conform to the esophagus and be flexible enough to allow for peristalsis.

Embodiment 19. The surgical mesh of embodiment 14, wherein the mesh material comprises polypropylene.

Embodiment 20. The surgical mesh of embodiment 14, wherein the mesh material comprises rough texture on one side.

### CONCLUSION

The various methods and techniques described above provide a number of ways to carry out the disclosure. Of course, it is to be understood that not necessarily all objectives or advantages described can be achieved in accordance with any particular embodiment described herein. Thus, for example, those skilled in the art will recognize that the methods can be performed in a manner that achieves or optimizes one advantage or group of advantages as taught herein without necessarily achieving other objectives or advantages as taught or suggested herein. A variety of alternatives are mentioned herein. It is to be understood that some embodiments specifically include one, another, or several features, while others specifically exclude one, another, or several features, while still others mitigate a particular feature by inclusion of one, another, or several advantageous features.

Furthermore, the skilled artisan will recognize the applicability of various features from different embodiments. Similarly, the various elements, features and steps discussed above, as well as other known equivalents for each such element, feature or step, can be employed in various combinations by one of ordinary skill in this art to perform methods in accordance with the principles described herein. Among the various elements, features, and steps some will be specifically included and others specifically excluded in diverse embodiments.

Although the application has been disclosed in the context of certain embodiments and examples, it will be understood by those skilled in the art that the embodiments of the application extend beyond the specifically disclosed embodiments to other alternative embodiments and/or uses and modifications and equivalents thereof.

In some embodiments, the terms “a” and “an” and “the” and similar references used in the context of describing a particular embodiment of the application (especially in the context of certain of the following claims) can be construed to cover both the singular and the plural. The recitation of ranges of values herein is merely intended to serve as a shorthand method of referring individually to each separate value falling within the range. Unless otherwise indicated herein, each individual value is incorporated into the specification as if it were individually recited herein. All methods described herein can be performed in any suitable order unless otherwise indicated herein or otherwise clearly contradicted by context. The use of any and all examples, or exemplary language (for example, “such as”) provided with respect to certain embodiments herein is intended merely to better illuminate the application and does not pose a limitation on the scope of the application otherwise claimed. No language in the specification should be construed as indicating any non-claimed element essential to the practice of the application.

Certain embodiments of this application are described herein. Variations on those embodiments will become apparent to those of ordinary skill in the art upon reading the foregoing description. It is contemplated that skilled artisans can employ such variations as appropriate, and the application can be practiced otherwise than specifically described herein. Accordingly, many embodiments of this application include all modifications and equivalents of the subject matter recited in the claims appended hereto as permitted by applicable law. Moreover, any combination of the above-described elements in all possible variations thereof is encompassed by the application unless otherwise indicated herein or otherwise clearly contradicted by context.

Particular implementations of the subject matter have been described. Other implementations are within the scope of the following claims. In some cases, the actions recited in the claims can be performed in a different order and still achieve desirable results. In addition, the processes depicted in the accompanying figures do not necessarily require the particular order shown, or sequential order, to achieve desirable results.

All patents, patent applications, publications of patent applications, and other material, such as articles, books, specifications, publications, documents, things, and/or the like, referenced herein are hereby incorporated herein by this reference in their entirety for all purposes, excepting any prosecution file history associated with same, any of same that is inconsistent with or in conflict with the present document, or any of same that may have a limiting affect as to the broadest scope of the claims now or later associated with the present document. By way of example, should there be any inconsistency or conflict between the description, definition, and/or the use of a term associated with any of the incorporated material and that associated with the present document, the description, definition, and/or the use of the term in the present document shall prevail.

In closing, it is to be understood that the embodiments of the application disclosed herein are illustrative of the principles of the embodiments of the application. Other modifications that can be employed can be within the scope of the application. Thus, by way of example, but not of limitation, alternative configurations of the embodiments of the application can be utilized in accordance with the teachings herein. Accordingly, embodiments of the present application are not limited to that precisely as shown and described.

What is claimed is:

1. A surgical mesh for the repair of hiatal hernias comprising:
a diaphragm portion sized for attachment to a diaphragm and comprises a large diameter component of a funnel;
an esophageal portion connected to the diaphragm portion, wherein the esophageal portion is sized for attachment to an esophagus and sized to form a narrow diameter component of a funnel; and
an opening that extends along the entire length of the diaphragm portion and the esophageal portion.

2. The surgical mesh of claim 1, wherein the diaphragm portion comprises a heavier gage material than the esophageal portion.

3. The surgical mesh of claim 1, wherein the diaphragm portion comprises a stiffer material than the esophageal portion.

4. The surgical mesh of claim 1, wherein the diaphragm portion and the esophageal portion are coated on one side with ePTFE.

5. The surgical mesh of claim 4, wherein the diaphragm portion and the esophageal portion are coated on other side with ePTFE.

6. The surgical mesh of claim 1, wherein the esophageal portion is configured to conform to the esophagus and be flexible enough to allow for peristalsis.

7. The surgical mesh of claim 1, wherein the esophageal portion and the diaphragm portion comprises polypropylene.

8. The surgical mesh of claim 1, wherein the esophageal portion comprises a rough texture on the side of the mesh that contacts the esophagus.

9. A method for implanting a mesh in a patient to address a hiatal hernia comprising:
providing a mesh comprising:
a diaphragm portion sized for attachment to a diaphragm and comprises a large diameter component of a funnel;
an esophageal portion connected to the diaphragm portion, wherein the esophageal portion is sized for attachment to the patient's esophagus and sized to form a narrow diameter component of a funnel; and
an opening that extends along the entire length of the diaphragm portion and the esophageal portion,
implanting the esophageal portion by wrapping the esophageal portion around the esophagus and thereby fixing the esophageal portion in place; and
implanting the diaphragm portion by positioning the diaphragm portion adjacent to the patient's diaphragm in an orientation that forms a funnel and fixing the esophageal portion in place.

10. The method of claim 9, wherein the implanting of the esophageal portion is performed separately from the implanting of the diaphragm portion.

11. The method of claim 10, wherein the esophageal portion and diaphragm portions are connected to each other after implantation.

12. The method of claim 9, wherein the esophageal portions and diaphragm portions are connected prior to implantation.

13. The method of claim 9, wherein the esophageal portion and the diaphragm portion comprises polypropylene.

14. A funnel shaped mesh for repairing hiatal hernias comprising:
a diaphragm portion configured for attachment to a diaphragm and sized to form a funnel mouth;
an esophageal portion connectable to the diaphragm portion, wherein the esophageal portion is sized for attachment to an esophagus and sized to form a tube part of the funnel; and
a seam that extends along the entire length of the diaphragm portion and the esophageal portion that is configured to allow the esophageal portion and the diaphragm portion to be wrapped around an esophagus,
wherein the diaphragm portion and the esophageal portion comprise a mesh material.

15. The surgical mesh of claim 14, wherein the diaphragm portion comprises a heavier gage material than the esophageal portion.

16. The surgical mesh of claim 14, wherein the diaphragm portion comprises a stiffer material than the esophageal portion.

17. The surgical mesh of claim 14, wherein the mesh material is coated on one side with ePTFE.

18. The surgical mesh of claim 14, wherein the esophageal portion is constructed to conform to the esophagus and be flexible enough to allow for peristalsis.

19. The surgical mesh of claim 14, wherein the mesh material comprises polypropylene.

20. The surgical mesh of claim 14, wherein the mesh material comprises rough texture on one side.
